(19) United States
(12) Patent Application Publication
(10) Pub. No.: US 2005/0106136 A1
(43) Pub. Date: May 19, 2005

(54) 7s immunoglobulin for treatment of choroidal neovascularisation

(76) Inventor: Richard Brunner, Neuenahr-Ahrweiller (DE)

Correspondence Address: SUTHERLAND ASBILL & BRENNAN LLP, 999 PEACHTREE STREET, N.E., ATLANTA, GA, 30309, US

(21) Appl. No.: 10/505,033

(22) PCT Filed: Feb. 15, 2002
(86) PCT No.: PCT/EP02/01638

(51) Int. Cl. A61K 39/395

(52) U.S. Cl. 424/130.1; 424/85.1

(57) ABSTRACT

The present invention refers to the use of 7 S immunoglobulin or an active fraction thereof for the manufacture of a medicament for the treatment of choroidal neovascularaisation and a pharmaceutical composition essentially consisting of an active fraction of 7 S immunoglobulin as pharmaceutically active compound and a method for the treatment of chorodial neovascularisation which comprises administering to the patient a preparation of 7 S immunoglobulin

### TECHNICAL FIELD OF THE INVENTION

The present invention refers to the use of immunoglobulins for the manufacture of a medicament for the treatment of choroidal neovascularisation and a pharmaceutical composition and a method for the treatment of choroidal neovascularisation.

### BACKGROUND OF THE INVENTION

Choroidal neovascularisation is a desease wherein new capillaries and blood vessels are formed in uncontrolled manner in the choroid of the eye.

Subretinal neovascularisations of the eye (choroidal neovascularisations [CNV]) lead to rapidly declining vision and loss of quality of life. At present the pathogenesis of the mechanism of CNV manifestation is not exactly known. The treatment of CNV is still a great ophthalmic-medical problem to be solved. Age-related macular degeneration is the leading cause of legal blindness in people over 60 years of age in the Western World (Kirchhof B., Deutsches Ärzteblatt 97 (21), (2000) A-1458-A-1462; Klaver et al., Arch. Ophthalmol. 116 (5) (1998) 653-658; Pauleikhoff, praktische Augenheilkunde 21 (2000) 59-65). The main reason for severe and progressive vision loss in AMD is the occurrence of subfoveal CNV (also termed wet or exsudative AMD) (Ferris, Arch. Ophthalmol. 102 (11) (1984) 1640-1642; Klaver et al., Arch. Ophthalmol. 116 (5) (1998) 653-658). Diseases such as pathologic myopia, ocular histoplasmosis syndrome, traumatic incidents or even inadequate laser treatment can cause CNV in younger patients (Cohen et al., Ophthalmology 102 (8) (1996) 1241-1244; Derosa et al., Doc. Ophthalmol. 91 (3) (1995) 207-222). Subretinal neovascular disease can also occur without any obvious reason as idiopathic CNV (Cleasby, Am. J. Ophthalmol. 81 (5) (1976) 590-599). At present the pathogenesis of the mechanism of CNV manifestation is not exactly known, although several angiogenetic factors have been identified as potential risk factors (Campochiaro, Mol. Vis. 5 (1999) 34; D'Amato, Ophthalmology 102 (9) (1995) 1261-1262; Zarbin, Eur. J. Ophthalmol. 8 (4) (1998) 199-206).

The treatment of CNV is still a great ophthalmic-medical problem to be solved. The aim is to prevent the expected progression as shown in the natural course of the disease. Improvement of the visual function is achieved only in a few cases.

State of Art:

Following treatments of choroidal neovascularisation have been discussed in the state of art:

Photodynamic Therapy

The primary mechanism of photodynamic therapy (PDT) is selective damage to fibrovascular choroidal tissue with preservation of overlying retinal structures such as photoreceptors and retinal pigment epithelium. The process of PDT involves the intravenous administration of a light-activated drug used as photosensitiser and its activation by a specific wavelength of light using a non-thermal diode laser device (Schmidt-Erfurth, Ophtalmologe 95 (10) (1998) 725-731; Scott, Drugs Aging 16 (2) (2000) 139-146). Verteporfin is the light-activated drug and active chemical component indicated for PDT (VIP Study Group 2001(a) Ophthalmology 108: 841-852; VIP Study Group 2001(b), Am. J. Ophthalmol. 131: 541-560).

The risk of an increased photosensitivity reaction after Verteporfin therapy due to sunlight exposure is declared to last only for two days (CIBAVision (1999) Visudyne product information CIBA Vision AG. 2-12-1999).

The efficacy of PDT in AMD patients with predominantly classic CNV has been proven in long-term results of a controlled, randomised study by the TAP Study Group (TAP Study Group (1999), Arch. Ophthalmol. 117 (10): 1329-1345; TAP Study Group (2001) Arch. Ophthalmol. 119 (2): 198-207, Bressler, IOVS 41 (5) (2000) 624-628). The recommended efficacy results in these patients prescribed less than 3 respectively 6 lines of visual acuity loss compared with placebo, or preservation of a mean of 2 lines of visual acuity and 2 segments of contrast sensitivity, compared with placebo.

Laser Treatment

As a result of the thermic effects of laser photocoagulation using argon- or cryptonlaser localised damage of the retinal photoreceptors and pigment epithelium is seen (Gloor, Schweiz. Rundsch. Med. Prax. 78 (16) (1989) 467-470; Wu, Cur. Opin. Ophthalmol. 10 (3) (1999) 217-220). Laser photocoagulation is used successfully in diabetic patients presenting with small and well-demarcated retinal mikrovascular abnormalities due to diabetes. In patients with subfoveal CNV only about 25% of the patients meet eligible treatment criteria for laser coagulation (MPS group, Arch. Ophthalmol. 109 (9) (1991) 1220-1241; Arch. Ophthalmol. 111 (9) (1993) 1220-1241; Arch. Ophthalmol. 112 (4) (1994) 489-499). The German Society of Ophthalmology published a statement on laser photocoagulation for subfoveal CNV saying that this method has not found world-wide acceptance for treatment of subfoveal CNV (Gabel, _{“}Ärztliche Behandlung”. 13-7-2000).

Radiation Therapy

According to the German Society of Ophthalmology radiation therapy for AMD has not proved efficacy in controlled clinical studies (Gabel, _{“}Ärztliche Behandlung”. 13-7-2000). Several controlled studies showed no or insufficient effects on the visual function after radiation therapy (RAD Study, Ophthalmology 12 (1999) 2239-2247; Anders et al., Ophthalmologe 11 (1998) 760-764; Tholen, Ophthalmologe 95 (10) (1998) 691-698). Furthermore the type or dose to be used for en effective radiation is not yet known. Therefore further controlled, randomised studies concerning radiation therapy in AMD are necessary (Archambeau et al., Int. J. Radiat. Oncol. Biol. Phys. 40 (5) (1998) 1125-1136; Berson, Semin. Radiat. Oncol. 9 (2) (1999) 155-162).

Pharmacological Therapy

No proven pharmacological treatment is known for subfoveal CNV. Neither for steroids (Challa, Aust. N. Z. J Ophthalmol 26 (4) (1998) 277-281), nor for the application of interferon alpha-2a (PTMD Study Group, Arch. Ophthalmol. 122 (5) (1997) 663-671) an efficacy could be proved. In AMD new therapeutic approaches using Gingko biloba (Clostre, Ann. Pharm. Fr. 57 Suppl 1 (1999) 18-88) and Phentoxyphyllin (Kruger et al., Arch. Ophthalmol. (1) (1998) 27-30) have been also tried.

New Approaches in Therapy of CNV

Further long-term results and controlled studies are needed to prove the efficacy of new therapeutic approaches in AMD such as submacular surgery, retinal translocation and retina transplantation (Ciulla et al., Surv. Ophthalmol. 43 (2) (1998) 134-146; Joffe, Int. Ophthalmol. Clin. 36 (2) (1996) 99-116).

State of Art Regarding 7 S Immunoglobulin

Intravenous immunoglobulin (IVIg), also called 7 S immunoglobulin, is increasingly used in the treatment of autoimmune and inflammatory diseases, including vasculitides and Kawasaki disease.

Intravenous gammaglobulin (7 S immunoglobulin) has been in use since 1981, primarily for prophylaxis in those with primary or secondary immunodeficiency states. Beneficial results have also been reported in the treatment of childhood idiopathic thrombocytopenic purpura, in CMV (cytomegalie virus) prophylaxis for bone marrow transplantation, amelioration of GVHD (graft versus host disease), and other autoimmune disorders. IVIg (7 S immunoglobulin) is known to contain antiidiotypic antibody activity against a number of autoantibodies (i.e. anti-ANA and anti-ANCA). IVIg (7 S immunoglobulin) is known to block antibody response in vivo and in vitro although the exact mechanisms are not known. IVIg (7 S immunoglobulin) has been used successfully to obtain improved post transfusion platelet-increments in refractory patients.

Intravenous immunoglobulin (7 S immunoglobulin) is therapeutic immunoglobulin (Ig) prepared from pools of plasma of several thousand healthy blood donors. In addition to its use as substitutive therapy for primary and secondary antibody deficiencies, IVIg (7 S immunoglobulin) exhibits immunomodulatory effects in diseases mediated by autoantibodies and in diseases believed to be primarily mediated by autoaggressive T cells in humans and in experimental animals. IVIg (7 S immunoglobulin) has been used effectively in the treatment of autoimmune cytopenias, the acute Guillain-Barré-syndrome, myasthenia gravis and anti-factor VIII autoimmune disease. Patients suffering from systemic inflammatory conditions such as dermatomyositis and, particularly Kawasaki syndrome greatly benefit from IVIg treatment. IVIg (7 S immunoglobulin) has also been used in the treatment of anti-neutrophil cytoplasmic antigen-associated systemic vasculitis. The mechanisms of action if IVIg are, as yet, poorly understood, although several mutually nonexclusive hypotheses have been proposed. These include the blockade of Fc γ receptors on phagocytic cells, interference with activated complement modulation of production and release of cytokines and their inhibitors, modulation of T- and B-lymphocyte functions, suppression of autoantibody production, and selection of immune repertoires.

U.S. Pat. No. 5,562,902 discloses a therapeutic method for inhibiting tumour metastasis and treatment of primary tumours comprising administering to a patient a preparation of intravenous gammaglobulin (IVIg).

U.S. Pat. No. 6,171,585 refers to methods of transplantation and to methods to immunosuppress a potential transplant recipient so as to be amenable to transplant with donor organs obtained from a variety of donors including histoincompatible donors. The method for transplanting an allograft in a patient comprises administering to the patient prior to transplantation an effective amount of an anti-HLA-antibody depleting agent which is essentially intravenous immunoglobulin.

The German patent application DE 199 00 503 A1 discloses the use of a composition for the manufacture of a medicament for the treatment of epidermal necrolysis, graft-versus-host disease, hepatitis, autoimmune thyroiditis, cancer or HIV.

On the cellular level the induction of neovascularisation, i.e. the formation of new blood vessels capillaries, may be explained as follows: The two major cellular components of the vasculature are the endothelial and smooth muscle cells. The endothelial cells form the lining of the inner surface of all blood vessels, and constitute a nonthrombogenic interface between blood and tissue. In addition, endothelial cells are an important component for the development of new capillaries and blood vessels. Thus endothelial cells proliferate during the angiogenesis, or neovascularization, associated with tumor growth and metastasis, as well as a variety of non-neoplastic diseases or disorders. During angiogenesis, or neovascularization endothelial cells proliferate, migrate and are responsible for tube formation of the future blood vessel or capillary.

The vascular endothelium is strategically located between the circulating blood and the vascular smooth muscle cells. Different agonists or stimuli transported by the circulating blood can trigger the endothelium to release potent relaxing (nitric oxide, prostacyclin, endothelium-derived hyperpolarizing factor) or contracting factors (endothelin, cycloxygenase products). These endothelium-derived vasoactive factors can modulate blood flow locally (Haefliger et al.; Prog. Retin. Eye Res. 20, (2001) 209-225).

Heterogeneity exists from one vascular bed to the other, or even between vessels, in the agonists able to stimulate the release of endothelium-derived vasoactive factors. In the ophthalmic circulation, nitric oxide and endothelin are strong vasoactive modulators. In many vascular diseases that are of importance in ophthalmology (hypercholesterolemia, arteriosclerosis, hypertension, diabetes, vasospastic syndrome, ischemia and reperfusion, choroidal and retinal neovascularisation, age related macular degeneration, diabetic retinopathy) the function of the endothelium can be impaired. Endothelial cells (ECs) are very heterogeneous cells that differ by acquisition and maintenance of specialized properties which is important for the functional homeostasis of different organs (Garlanda C, Dejana E: “Heterongenity of endothelial cells. Specific markers.” In Arterioscler. Thromb. Vasc. Biol. 17, (1997) pp 1193-1202).

Endothelial cells also have site-specific differences in the sensitivity to cell injury (Murphy et al. Heterogeneity of vascular endothelial cells: differences in susceptibility to neutrophil-mediated injury; Microvasc. Res. 56, (1998), pp 203-211). Especially in the eye, alteration of the blood-retina barrier have important consequences on eye functional integrity. Even in the eye ECs and blood vessels from retina and choroid differ largely. For instance in the rat, choroidal arterioles are much larger in diameter than retinal arterioles which may explain differences of hemodynamics of both tissues (Ninomiya H, Kuno H; Vet. Ophthalmol. 4, (2001) pp 55-59). In addition ECs from the choriocapillaris are fenestrated whereas retinal ECs are not. The fenestrated capillaries in the choroid are very permeable to low molecular weight substances; sodium permeability in the choroid is probably 50 times that in skeletal muscle (Tornquist P, Alm A, Bill A; Eye 4 (Pt 2), (1990) pp 303-309). These results in high concentrations and rapid turnover of nutrients in the extra-vascular compartment of the choroid. Also the retinal capillaries, with tight junctions between the endothelial cells, have very low permeability even to sodium in contrast to the choriocapillaris.

Biochemical variations, such as the expression of ion channels, connexin subtypes and other important components of second messenger cascades, have been documented in the smooth muscle and endothelial cells in different parts of the body (Hill C E, Phillips J K, Sandow S L (2001): Med. Res. Rev. 21 (2001), 1-60). Anatomical variations, in the presence and prevalence of gap junctions between smooth muscle cells, between endothelial cells and at myoendothelial gap junctions, between the two cell layers, have also been described. These factors will contribute further to the heterogeneity in local and conducted responses (Hill C E, Phillips J K, Sandow S L; Med. Res. Rev. 21, (2001), pp 1-60).

There exist different drugs that can modulate the vasoactive function of the vascular endothelium. In other words, it appears that the vascular endothelium plays an important role in both the physiology and pathophysiology of the regulation of blood flow. The modulation of this regulatory system by different drugs might open new therapeutical approaches to treat vascular disorders in ophthalmology. It has been shown that immunoglobulin can inhibit one parameter of neovascularization, which is proliferation in human umbilical vein endothelial cells in vitro (Xu C. et al.: Modulation of endothelial cell function by normal polyspecific human intravenous immunoglobulines. Am. J. Pathol. 153, (1998), 1257-1266). Xu et al. describes that by using human umbilical vein endothelial cells (HUVECs) as target cells, IVIg (7 S immunoglobulin) from different commercial sources modulates the function of endothelial cells (ECs). In their studies IVIg (7 S immunoglobulin) inhibited EC proliferation in a dose- and time-dependent manner. It was also shown that IVIg down-regulated the TNF-α or IL-1β-induced expression of mRNA encoding major adhesion molecules, chemokines, and proinflammatory cytokines, which are significantly implicated in the leukocyte recruitment observed in several inflammatory diseases. However, these cells (human umbilical vein endothelial cells) are not an ideal model, since they are close to senescence and are cultured from hypoxic and possibly activated vessels (Garlanda C, Dejana E; Arterioscler. Thromb. Vasc. Biol. 17, (1997), pp 1193-1202). Therefore, it is preferable to culture the endothelium from the microvasculature of the target organ and to maintain their specialized properties in vitro.

Pathological neovascularization is not only characterized by proliferation but also by migration and tube formation of endothelial cells. The effect on migration and tube formation of endothelial cells by immunoglobulin has not yet been shown. The effects of immunoglobulin on endothelial cells from the eye are completely unknown.

In view of the state of art, there did exist a need agents showing inhibitory activities on choroidal endothelial cells and choroidal neovascularisation. There also exist a need for medicaments and methods for the treatment of choroidal neovascularisation (CNV).

Therefore it was technical object of the present invention to provide a medicament for the treatment of choroidal neovascularisation.

### SUMMARY OF THE INVENTION

The present invention generally solves the problems referred to above by providing the use of 7 S immunoglobulin or an active fraction thereof for the manufacture of a medicament for the treatment of choroidal neovascularisation.

In particular, it is provided the use of 7 S immunoglobulin or an active fraction thereof for the manufacture of a medicament for the treatment of choroidal neovascularisation, said 7 S immunoglobulin or said active fraction thereof having the ability to inhibit one or more activities selected from the group consisting of:

a) choroidal neovascularisation,

b) proliferation of choroidal endothelial cells,

c) migration of choroidal endothelial cells,

d) tube formation of choroidal endothelial cells.

### DETAILED DESCRIPTION OF THE INVENTION

Definitions

As used herein “gamma globulin” or “7 S immunoglobulin” is the serum globulin fraction that is mainly composed of IgG molecules.

As used herein, “IVIg” or “intravenous immunoglobulins” or “7 S immunoglobulin” refers to a gamma immunoglobulin fraction which may be prepared by fractional alcohol precipitation (such as according to Cohn-Oncley-method) from human blood plasma, such as those 7 S gamma immunoglobulin preparations commercially available from several sources. The 7 S immunoglobulin fraction again essentially consists of the immunoglobulins IgG₁, IgG₂, IgG₃ and IgG₄.

As used herein, _{“}active” fraction of 7 S immunoglobulin refers to pharmaceutically activity in terms of being effective in the treatment of choroidal neovascularisation. The term “active” does also refer to a biological (and pharmaceutically) activity as being effective in inhibiting choroidal neovascularisation, inhibiting proliferation of choroidal endothelial cells, inhibiting migration of choroidal endothelial cells, inhibiting tube formation of choroidal endothelial cells.

Intravenous immunoglobulins (7 S immunoglobulin) are therapeutic preparations of normal polyspecific IgG obtained from plasma pools of over 6,000 healthy blood donors. Currently used preparations are made of intact IgG with a distribution of subclasses corresponding to that of normal serum and have a half-life of three weeks in vivo for IgG₁, IgG₂ and IgG₄, and somewhat less for IgG₃. Most of the preparations contain only traces of IgA, IgM and of Fc-dependent IgG aggregates (see Kaveri et al., in Clin. Exp. Immunol. 86 (1991) 192-198).

As used herein “fragments” of IVIg or gamma globulin or 7 S immunoglubulin are portions of intact immunoglobulins such as Fc, Fab, Fab′, F(ab′)₂ and single chain immunoglobulins.

7 S immunoglubulin preparations contain up to 30% (w/w) of F(ab′)₂-F(ab′)₂ dimers as assessed by size-exclusion chromatography and electronmicroscopy. The dimers are the consequence of V-region complementarity between immunoglobulins in the pool (see Roux & Tankersley, in J. Immunol. 134 (1990) 1387). Owing to the large number of donors, IVIg (7 S immunoglobulin) represent a wide spectrum of the expressed normal human IgG repertoire, including antibodies to external antigens, autoreactive antibodies and anti-antibodies.

Commercial IVIg (7 S immunoglobulin) preparations are widely available, for example, from Aventis Behring, Cutter Laboratories, MedImmune; Novartis Pharma (Nürnberg, Germany), Octapharma, Venoglobulin, Miles Inc. (West Haven, Conn.), N.V. Baxter S.A. (Lessines, Belgium), Sandoz Pharma Ltd. (Basle, Switzerland), Instituto Sierovaccinogeno Italiano (Isiven, Italy) and Jackson Immunoresearch Laboratories, Inc. (West Grove, Pa.). The commercially available IVIg (7 S immunoglobulin) preparations contain mainly IgG molecules and in maltose or glycine carriers. Also contemplated for use herein are aqueous solutions containing higher concentrations of IVIg (7 S immunoglobulin), such as approximately 25% (w/w)-75% (w/w). Substantially pure preparations of the “IgG-fraction of IVIg” are also suitable for use herein. Substantially pure IgG-fractions typically contain greater than 50% (w/w) of an IgG-fraction, preferable greater than 75% (w/w), and most preferably greater than 95% (w/w) of an IgG-fraction. Such substantially pure IgG-fractions are commercially available from several sources.

According to a National Institutes of Health (NIH) Consensus Conference report, the incidence of adverse side effects associated with IVIg use in humans, used at dosage regimens comparable to the ones contemplated by the present invention, is usually less than 5% with most of those reactions being “mild and self-limited”. The report adds that “severe reactions occur very infrequently and usually do not contraindicate further IVIg therapy”. the NIH report also notes that “[n]either HIV nor hepatitis B infection has been transmitted to recipients of products currently licensed in the United States”. NIH Consensus Conference, “Intravenous Immunoglobulin: Prevention and Treatment of Disease”, JAMA, 264, pp. 3189-3193 (1990).

The 7 S immunoglubulin preparations that may be used according to the present invention include commercially available preparations of intact 7 S immunoglubulin and preparations of the F(ab′)₂ fragments of 7 S immunoglubulin. Recombinantly produced gamma globulin and their fragments may also be used according to this invention. The use of recombinant single chain antibodies is also envisioned.

The dosage of 7 S immunoglubulin and the method of administration will vary with the severity of the particular condition being treated, the duration of treatment, the adjunct therapy used, the age and physical condition of the subject of treatment and like factors within the specific knowledge and expertise of the treating physician. However, singe dosages for intravenous and intracavitary administration can typically range from 400 mg to 2 g per kilogram body weight, preferably 2 g/kg (unless otherwise indicated, the unit designated “mg/kg” or “g/kg”, as used herein, refers to milligrams or grams per kilogram of body weight). The preferred dosage regimen is 400 mg/kg/day for 5 consecutive days per month or 2 g/kg/day once a month. According to the present invention 7 S immunoglubulin was found to be effective in the treatment of choroidal neovascularisation when administered by intravenous or intraperitoneal injection and in the dose range of 500-1000 mg/kg/week.

In an other embodiment of this invention, the 7 S immunoglubulin preparation is administered via the subcutaneous route. The typical dosage for subcutaneous administration can range from 4 mg to 20 mg per kg body weight. According to the present inventions 7 S immunoglubulin was found to be effective in the treatment of choroidal neovascularisation administered subcutaneously in the dose 500-1000 mg/kg/week.

According to the present invention 7 S immunoglubulin may be administered as a pharmaceutical composition containing a pharmaceutically acceptable carrier. The carrier must be physiologically tolerable and must be compatible with the active ingredient. Suitable carriers include sterile water, saline, dextrose, glycerol and the like. In addition, the compositions may contain minor amounts of stabilising or pH buffering agents and the like. The compositions are conventionally administered through parenteral routes, with intravenous intracavitary or subcutaneous injection, being preferred.

Surprisingly it has been found that intravenous 7 S immunoglubulin is effective in the treatment of choroidal neovasularisation (FIGS. 4 to 8). Administration of intravenous 7 S immunoglubulin resulted in a significant improvement of visual acuity.

The inventors of the present invention carried out studies by using 7 S immunoglobulines for treatment of CNV in young patients (diagnosis e.g. idiopathic CNV, presumed ocular histoplasmosis syndrome [POHS]) and in older patients (diagnosis e.g. age-related macular degeneration [AMD]). The treatment was performed in repeated therapeutic cycles and 7 S immunoglobulin was given intravenously. The efficacy results have been proved by the change of the visual acuity with ETDRS-charts (Early Treatment Diabetic Retinopathy Study, Lighthouse, N.Y.). The change of the visual acuity is measured in lines on the EDTRS-chart, whereby a change of one line corresponds to a change of 0.1 LogMAR units. The ETDRS-charts have been developed especially for patients with maculopathy (Ferris et al. 1982).

Intravenous 7 S immunoglubulin does also inhibit the proliferation (FIGS. 1 and 2), migration and tube formation of choroidal endothelial cells in cell culture.

Although Xu et al. (Am. J. Pathol. 153 (4) (1998), pp. 1257-1266) have found that IVIg inhibited proliferation of endothelial cells, these results could not be transferred to the choroidal endothelial cells. Xu et al. did show this effect on human umbilical vein endothelial cells (HUVECs). However, it is known in the art that endothelial cells of different tissues show a strong heterogeneity.

Garland C. and Dejana E. summarise the observations made on the heterogeneity of endothelial cells in their review article “Heterogeneity of Endothelial Cells” in Arteriosclerosis, Thrombosis and Vascular Biology (1997; 17:1193-1202). In their review article the authors describe that the endothelium is considered a sparse organ system, due to its vast extension and ability to exert a complex array of specialised functions. A unique characteristic of endothelial cells (ECs) is that, although they present many common functional and morphological features, they also display remarkable heterogeneity in different organs. Even in the same organ, the endothelium of large and small vessel, veins and arteries exhibits significant heterogeneity. An extreme case is the kidney, which contains different types of ECs: fenestrated in the peritubular capillaries, discontinuous in glomerular capillaries and continous in other regions. Embryonic ECs seem particularly “plastic.” Most of the specialised characteristics of ECs are induced during development, whereas adult endothelium is not equally susceptible to differentiation factors. Despite its stable constitutive properties, the adult endothelium can reversibly change its functions on activation. Adult ECs can be reprogrammed according to the transitory needs of the organism. For instance, exposure of ECs to inflammatory cyokines, such as IL-1 and tumour necrosis factor, or to growth factors, such as VEGF or FGF, induces a complex functional reprogramming, which implies the neosynthesis of some genes and the repression of others. ECs can be activated several times during their life span by the same of different cytokines and thereby display different and reversible phenotypes.

As mentioned above endothelial cells (ECs) display remarkable heterogeneity in different organs and even in the same organ, the endothelium of large and small vessel, veins and arteries exhibits significant heterogeneity. The inventors of the present invention performed studies with human umbilical vein endothelical cells (HUVECs), retinal endothelical cells and choroidal endothelial cells. 7 S immunoglobulin did show inhibitory effects on choroidal endothelial cells and HUVECs. However, it did not show any effects on retinal endothelial cells at all (see FIG. 3). Since endothelial cells from the same organ (from retina and choroid, respectively) show such extremely different sensitivity to 7 S immunoglobulin the person skilled in the art would not have expected that 7 S immunoglobulin could be used as a medicament for the treatment of choroidal neovascularisation.

In a preferred embodiment said active fraction is selected from the group consisting of IgG fractions of 7 S immunoglobulin IgG₁, IgG₂, IgG₃, IgG₄ and mixtures thereof.

The distribution of the subclasses of 7 S immunoglobulin in the medicament may correspond to the one in the blood serum of a healthy person. Preferably the distribution of the IgG subclasses in the medicament is as follows:
IgG₁: 60-70% (w/w)
IgG₂: 25-30% (w/w)
IgG₃: 3-8% (w/w)
IgG₄: 0.5-3% (w/w).

In a further preferred embodiment said 7 S immunoglobulin is present in the medicament as a composition comprising at least 50% (w/w), preferably at least 80% (w/w), more preferred at least 95% (w/w) gamma immunoglobulin.

In a further preferred embodiment said active fraction of 7 S immunoglobulin is present in the medicament in an amount of at least 50% (w/w), preferably of at least 80% (w/w), more preferred of at least 95% (w/w).

Preferaby said active fraction is composed of fragments of gamma immunoglobulin. The medicament which is used for the treatment of choroidal neovascularisation may contain fragments of gamma immunoglobulin which are selected from the group consisting of F(ab′)₂, Fab′, Fab and Fc of gamma immunoglobulin.

In yet another embodiment the medicament will contain gamma immunoglobulin in an amount effective to inhibit choroidal neovascularisation. Particularly, the gamma immunoglobulin will be present in the medicament in an amount effective to inhibit the growth of new blood vessels in the eye. In a further preferred embodiment the gamma immunoglobulin is present in the medicament in an amount effective to inhibit the proliferation of choroidal endothelial cells. In a further preferred embodiment the gamma immunoglobulin is present in the medicament in an amount effective to inhibit migration of choroidal endothelial cells and effective to inhibit tube formation which is initiated by choroidal endothelial cells when new blood vessels develop.

The present invention also solves the problems referred to above by providing a Pharmaceutical composition essentially consisting of an active fraction of 7 S immunoglobulin as pharmaceutically active compound.

Preferably, said active fraction in the pharmaceutical composition has the ability to inhibit one or more activities selected from the group consisting of:

a) choroidal neovascularisation,

b) proliferation of choroidal endothelial cells,

c) migration of choroidal endothelial cells,

d) tube formation of choroidal endothelial cells.

In a further preferred embodiment said active fraction is one, two or three of the components selected from the group consisting of IgG fractions of 7 S immunoglobulin IgG₁, IgG₂, IgG₃, IgG₄.

In another preferred embodiment said active fraction of 7 S immunoglobulin is present in the pharmaceutical composition in an amount of at least 50% (w/w), preferably of at least 80% (w/w), more preferred of at least 95% (w/w).

The active fraction preferably is composed of fragments of gamma immunoglobulin, more preferred the fragments of gamma immunoglobulin are selected from the group consisting of F(ab′)₂, Fab′, Fab and Fc of gamma immunoglobulin.

The active fraction contained in the pharmaceutical composition of the present invention is contained in an amount effective to inhibit choroidal neovascularisation and growth of new blood vessels in the eye. Furthermore, the active fraction is contained in an amount effective to inhibit the proliferation of choroidal endothelial cells, the migration of choroidal endothelial cells, and the tube formation initiated by choroidal endothelial cells.

The present invention also solves the problems referred to above by providing therapeutic methods for the treatment of choroidal neovascularisation which comprises administering to the patient a preparation of 7 S immunoglobulin or an active fraction thereof.

Preferably, said 7 S immunoglobulin or said active fraction of 7 S immunoglobulin is administered in an amount effective to inhibit choroidal neovascularisation. In a further preferred method for the treatment of choroidal neovascularisation, said 7 S immunoglobulin or an active fraction thereof have the ability to inhibit one or more activities selected from the group consisting of:

a) choroidal neovascularisation,

b) proliferation of choroidal endothelial cells,

c) migration of choroidal endothelial cells,

d) tube formation of choroidal endothelial cells.

Further embodiments of the method of the present invention comprise features mentioned above in reference to the use of 7 S immunoglobulin for the manufacture of the medicament for the treatment of choroidal neovascularisation.

The present invention will be explained in more detail by the following examples and with reference to the FIGS. 1 to 8, which are not to be construed as limiting the scope of the present invention in any manner.

### BRIEF DESCRIPTION OF THE FIGURES

Figure Legends

FIG. 1 shows the inhibition of choroidal endothelial cell proliferation by IgG (7 S immunoglobulin) from 2 independent experiments after 48 hours. Proliferation is inhibited in a concentration dependent manner. This experiment was performed to find the range of efficient dosage.

FIG. 2 shows the inhibition of choroidal endothelial cell proliferation by IgG (7 S immunoglobulin) from 4 independent experiments, that are different from the experiment presented in FIG. 1, after 48 hours in comparison to an untreated control. Proliferation is inhibited in a concentration dependent manner.

FIG. 3 shows the effect of 40 mg/ml IgG (7 S immunoglobulin) on EC migration after 8 hours depending on the cells origin. Migration is inhibited in choroidal ECs by 56% and by 35% in HUVECs. The inhibition of IgG is absent in retinal ECs.

FIG. 4 shows the change of visual acuity in 10 patients with idiopathic CNV or POHS over a period of 18 months after treatment with 7 S immunoglobulin. After 12 months 9/10 eyes and after 18 months 8/10 eyes improved in visual acuity.

FIG. 5 shows the change of visual acuity of these eyes after 3 and 18 months presented as box-plots with the corresponding median after treatment with 7 S immunoglobulin. The improvement is significant.

FIG. 6 shows the change of visual acuity in a young male patient with idiopathic CNV in the right eye (OD). The period of time of treatment with 7 S immunoglobulin is marked by an arrow (Treatment).

FIG. 7 shows the change of visual acuity in a young female patient with POHS in both eyes (right eye: OD, left eye: OS). The period of time of treatment with 7 S immunoglobulin is marked by an arrow (Treatment).

FIG. 8 shows the change of visual acuity in a 70 year old patient with CNV caused by age-related macular degeneration (AMD) after treatment with 7 S immunoglobulin. The stars mark the change of visual acuity before a therapeutic cycle. Before treatment the visual acuity on the better right eye (OD) was 0.125.

### EXAMPLES

Methods

Preparation of Primary Human Choroidal or Retinal Endothelial Cells

Human eyes were obtained within 30 h of death from the donors. They were free of known ocular diseases. The eyes were dissected, and the anterior segment, vitreous and retina were separated. The choroid with retinal pigment epithelium layer or the retinas were incubated for 30 min at 37° C. with Accutase (PAA). Then the retinal pigment epithelium cells were removed with a spatula. The choroid was stripped off the sclera, washed with Hanks' balanced salt solution (HBSS) and then incubated with 0.25% trypsin and 0.02% EDTA at 37° C. for 1 h. The choroidal or retinal fragments were further incubated with HBSS containing collagenase 4000 U/ml (1:4 in HBSS) for 30 min at 37° C., washed twice with HBSS and further incubated with collagenase for 2 h at 37° C. Then the choroidal or retinal fragments were passed through sterile mesh (70 μm pore size), the suspension centrifuged (5 min at 1500 rpm) and the supernatant discarded. The pellet was washed with HBSS containing 1% BSA and once again centrifuged. The cell pellet was resuspend in 1 ml HBSS (1% BSA) and transferred to 1.5 ml eppendorf tubes. For separation of the endothelial cells magnetic beads (Dynabeads CD-31, dynal, Cat. N 111.28) precoated with a IgG1 monoclonal antibody (clone 9G11) specific for the human CD31 cell surface antigen were used. The magnetic beads were washed several times with 1% BSA in HBSS and then 10 μl of the beads solution (1×107 beads per 1 ml cell suspension) were added to the cell suspension and mixed gently for 2 h at room temperature. After that, the tubes were placed on Dynal Magnetic particle Concentrator (Dynal MPC®) and allowed to sediment for 2 min. The supernatants were removed and the cells attached to the magnetic beads were washed with 1% BSA in HBSS, resuspended in endothelial cell growth medium (Promocell, cat. N 22020) containing 0.4% ECGS/H, 5% FCS, 10 ng/ml EGF, 1 ng/ml hydrocortison, 50 ng/ml bFGF, 50 ng/ml Amphotericin B, 50 μg/ml Gentamicin and seeded on culture dishes.

Human umbilical vein endothelial cells (HUVECs) were bought from PromoCell (Heidelberg, Germany).

Immunocytochemistry

The purity of the cell preparation was determined by endothelial cell specific antibodies. For immunocytochemistry, the cells were seeded on immunochambres at a density of 5×10³ cells/chamber and cultured for 5 days in endothelial cell growth medium. After fixation with 4% paraformaldehyde at 4° for 20 min, cells were washed three times with phosphate buffered saline (PBS). To block the unspecific bindings, the cells were incubated for 1 h with 5% BSA in PBS. After washing, the cells were reacted with anti-human Von Willebrand factor antigen rabbit polyclonal, dilution 1:200 in PBS, with 5% rabbit serum, Dako, Cat. N A0082) overnight at 4° C. The cells were washed five times with PBS and then incubated with a secondary antibody conjugated with alkaline phosphatase (anti-rabbit IgG, dilution 1:400 in PBS, 1% sheep serum, Sigma) for 2-3 h at room temperature, washed four times with PBS and exposed to new fuchsin for 5 min. Hemalun contrast staining was performed. Then the cells were washed and mounted with Kaisers Glyceringelatine and observed by light microscopy. As a negative control, normal rabbit serum (diluted 1:700 in PBS) was used instead of the primary antibody.

Statistical Analysis:

For statistical analysis Student's t-test was used.

### Example 1: Cell Proliferation Assay

Choroidal endothelial cells were seeded on 96-well culture plates (Nunc) at a density of 1000 cells/well and cultured in endothelial cell growth medium (5% FCS) for 24 h. The medium was discarded, the cells were washed three times with PBS and exposed to basal endothelial cell medium only (control) containing 50 ng/ml VEGF (positive control). The cells were exposed to 5 mg, 10 mg, 20 mg and 40 mg immunoglobulin, respectively (Sandoglobulin®, Novartis Pharma, Nürnberg, Germany).

WST-1 assay was used to determine the proliferation rate of endothelial cells according to the manufacturer's instructions (Boehringer Mannheim). The colorimetric proliferation assay is based on the cleavage of the tetrazolium salt WST-1 by mitochondrial dehydrogenases in viable cells. The most effective dose of VEGF was determined by the proliferation assay in a pilot study on days 1, 3, and 5 after plating the endothelial cells.

Results:

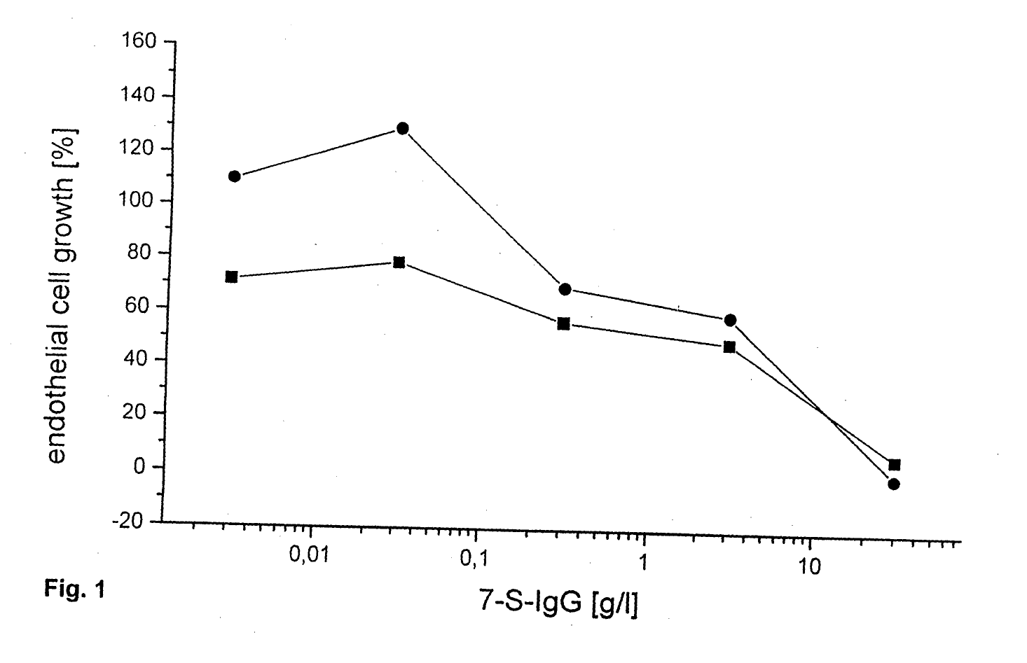

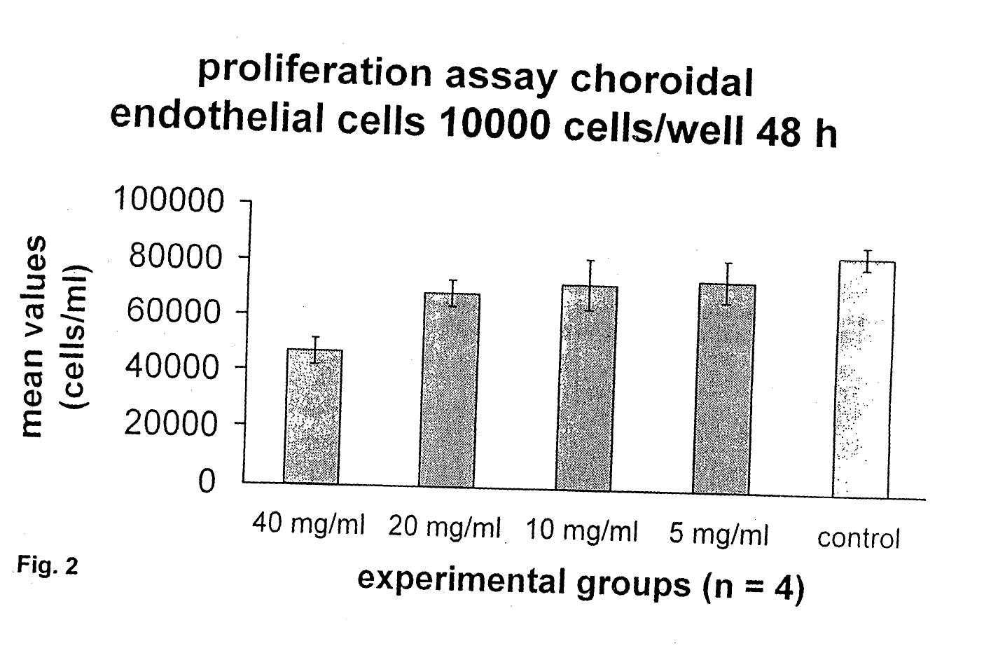

The stimulatory effect of VEGF (50 ng/ml) on CEC proliferation was significantly blocked after 48 hours in a concentration dependent manner by exposure of the cells to 20 or 40 mg IG/ml (n=4) by 33% (p=0.004) or 45% (p=0.00004). The results are shown in FIGS. 1 and 2.

### Example 2: Migration Assay

Migration of choroidal, retinal and umbilical vein ECs was assayed using FluoroBlock Inserts according to the manufacturer instructions (Falcon). The assay is based on light-tight polyethylene terephthalate (PET) microporous membrane (8 μm pore size) which are constructed to specifically detect fluorescence of labeled cells and molecules below the insert. The membranes were coated with gelatin (0.1% in 0.1 M PBS) for 1 h at a 37° C. Then the inserts were exposed to 1) medium+50 ng/ml VEGF (positive control); 2) medium+50 ng/ml VEGF containing 40 mg/ml Immunoglobulin; ECs (passages 2 to 4) were serum-starved (DMEM, 0.5% FCS) for 3 h, collected with 0.02% EDTA and loaded into the membranes of inserts (5×10³ cells/per membrane). After 8 h of incubation at 37° C., the filters were removed, washed with 0.1% PBS and fixed in 4% paraformaldehyde (in 0.1% PBS) for 30 min at room temperature. The filters were washed again with PBS and the cell nuclei were stained with 4′,6-diamidino-2-phenylindole (DAPI) producing a blue fluorescence (460 nm). Further, the filters were mounted with Vectashield Mounting medium (Vector Lab) bottom side up on glass slide. The numbers of cells in three different microscopic fields (×10 objective) were counted. The net number of migrated cells was obtained by subtracting the number of migrated cells in the absence of chemoatractant from that in the presence of such a stimulant.

Results:

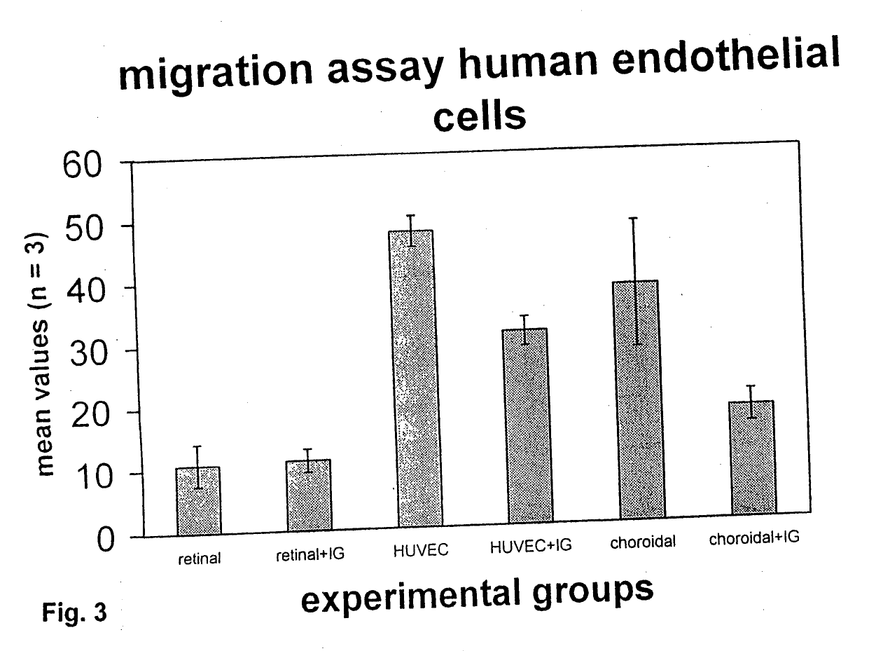

The effect-of IG on ECs migration after 8 hours was different and depended on the cells origin. Migration was inhibited in choroidal ECs by 56% and by 35% in HUVECs. The inhibition by IG was absent in retinal ECs (FIG. 3).

### Example 3: In Vitro Tube-Formation Assay

Growth factor depleted Matrigel (Harbor extracellular matrix basement membrane), Tebu, cat. N 2001) was applied into a 24-well tissue culture plate (400 μl/well) and forms an in vitro basement membrane. After polymerization of the Matrigel (37° C., 1 h), primary human choroidal endothelial cells were seeded on the top of the gels at a density of 10 000 cells/well and cultivated in endothelial cell growth medium containing 5% FCS for 24 h. Then the medium was aspirated and the cells were exposed to: 1) basal endothelial cell medium (contains no growth factors, 2% FCS, Promocell cat. N 22210) (control); 2) basal endothelial cell medium containing 50 ng/ml VEGF; 3) conditioned medium (CM) (keratinocyte SF, Gibco Cat. N) from Ad.PEDF-infected primary rat IPE cells; 4) CM from AD.PEDF-infected rat IPE cells+50 ng/ml VEGF; 5) CM from Ad.eGFP-infected rat IPE cells; 6) CM from Ad.eGFP-infected rat IPE cells+50 ng/ml VEGF; 7) CM from non-infected rat IPE cells. The plate was incubated at 37° C. for 24 h, and then the medium was aspirated and cells were fixed in neutral buffered 10% formalin. The gel proteins allow cell alignment and tube formation, which can be seen under an inverted light microscope. Representative pictures were taken at ×10 magnification.

Results:

Tube formation by CEC was apparent in all experimental groups but was less prominent in the presence of 40 mg/ml IG.

Conclusion:

Blockade of cellular proliferation, migration and tube formation may explain and confirm the therapeutic effect by 7 S immunoglobuline on choroidal neovascularisation described below in example 4.

### Example 4: Treatment of Patients Suffering from CNV

Studies were carried out by using 7 S immunoglobulines for treatment of CNV in young patients (diagnosis e.g. idiopathic CNV, presumed ocular histoplasmosis syndrome [POHS]) and in older patients (diagnosis e.g. age-related macular degeneration [AMD]).

The treatment was performed in therapeutic cycles over a period of 3-5 days, which were repeated according to the clinical course. A total amount of 50-100 g 7 S immunoglobulines were given intravenously in a therapeutic cycle. The heart and circulation parameters were surveyed during and after the infusion. The efficacy results have been proved by the change of the visual acuity with ETDRS-charts (Early Treatment Diabetic Retinopathy Study, Lighthouse, N.Y.). The change of the visual acuity is measured in lines on the EDTRS-chart, whereby a change of one line corresponds to a change of 0.1 LogMAR units. The ETDRS-charts have been developed especially for patients with maculopathy (Ferris et al. New visual acuity charts for clinical research; Am. J. Ophthalmol.; 94 (1982) pp 91-96). Besides the clinical evaluation of the retina fundusphotographs and fluorescein-angiographies were performed for documentation.

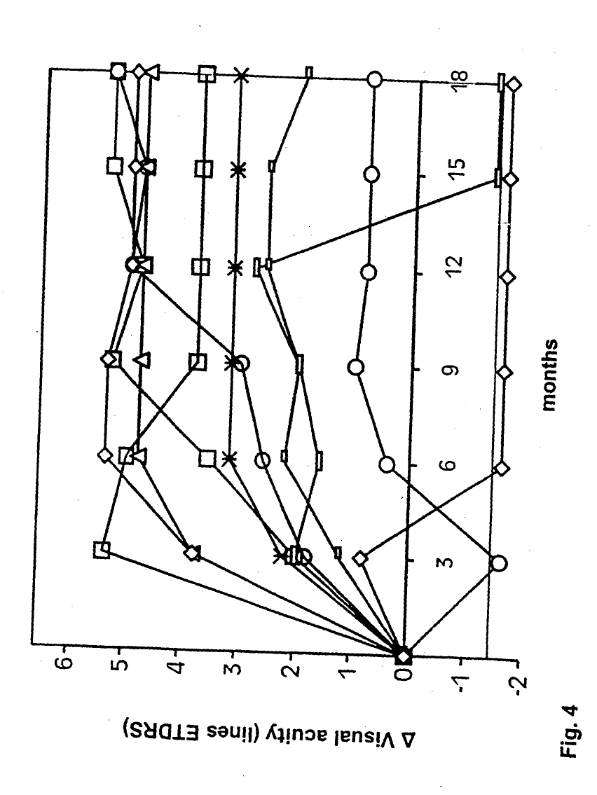

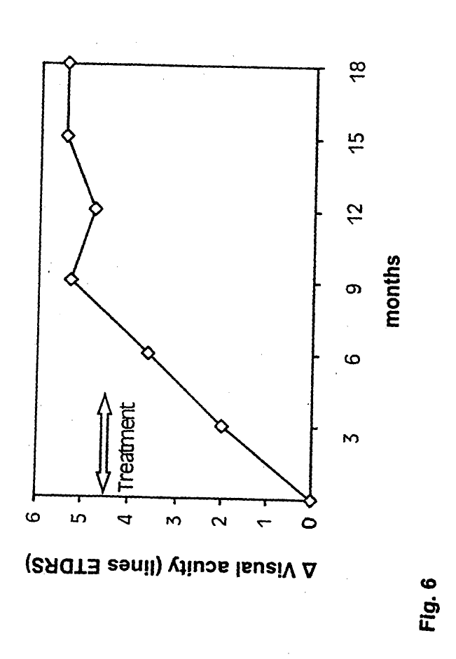

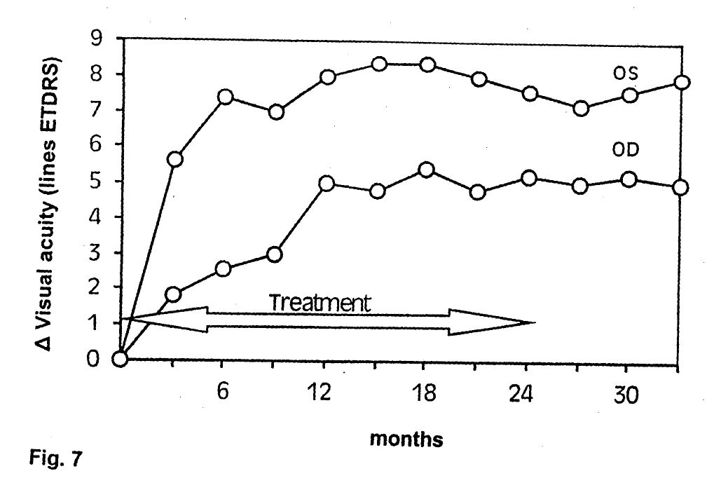

10 younger patients with an idiopathic CNV or POHS were treated over a mean period of 18 month by repetitive infusion of intravenous 7 S immunoglobulins. The mean age of the patients was 38.5 years. The treatment was applied every 3 to 6 weeks at an individual base. A total dose of 50 to 100 g was administered during each treatment cycle. Clinical endpoint of the study was visual acuity measured by ETDRS charts. The results are summarized in FIGS. 4 to 8.

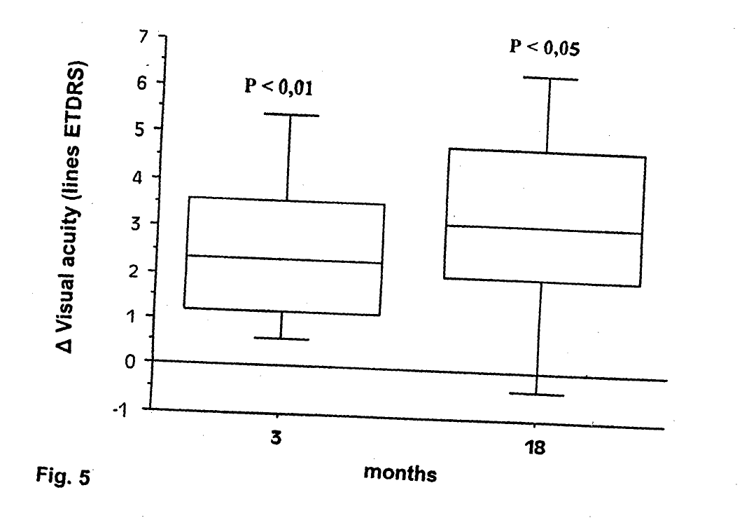

After the trial 9 out of 10 patients showed an improvement of visual acuity. The mean improvement was 2.3 lines (p<0.01) after three and 3.2 lines (p=0.02) after 18 months. According to the fluorescein angiography a partial involution and scaring of the CNV was observed. The study demonstrated the beneficial effect of intravenous 7 S immunoglobulins on the natural course of idiopathic CNV. The repetitive treatment led to functional and morphological improvements. The results are summarized in FIG. 5.

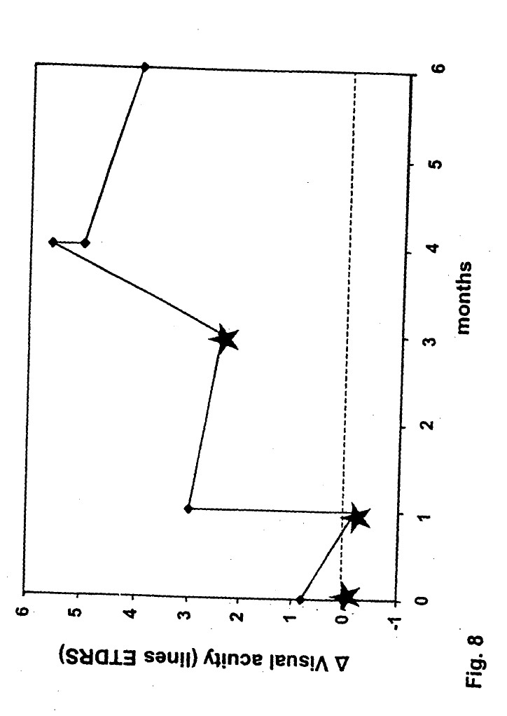

As example for an older patient FIG. 8 summarizes the results of the treatment by showing the change of visual acuity in a 70 year old patient with CNV caused by age-related macular degeneration (AMD).

The treatment was tolerated well in general. The ophthalmologic clinical course showed generally a reduction of exsudation, a reduction of progression of CNV and transition into a non-active stadium of scarring.

Conclusion:

The clinical results show a positive effect of a treatment with 7 S immunoglobulines on the natural course of CNV.

1-79. (canceled)

80. Use of 7 S immunoglobulin or gamma globulin or a serum globulin fraction that is mainly composed of IgG molecules or intravenous immunoglobulin for the manufacture of a medicament for the treatment of choroidal neovascularisation.

81. Use of an active fraction of 7 S immunoglobulin or gamma globulin or a serum globulin fraction that is mainly composed of IgG molecules or intravenous immunoglobulin for the manufacture of a medicament for the treatment of choroidal neovascularisation.

82. Use of 7 S immunoglobulin or gamma globulin or a serum globulin fraction that is mainly composed of IgG molecules or intravenous immunoglobulin for the manufacture of a medicament for the treatment of choroidal neovascularisation, said 7 S immunoglobulin or gamma globulin or a serum globulin fraction that is mainly composed of IgG molecules or intravenous immunoglobulin having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells.

83. Use of an active fraction of 7 S immunoglobulin or gamma globulin or a serum globulin fraction that is mainly composed of IgG molecules or intravenous immunoglobulin for the manufacture of a medicament for the treatment of choroidal neovascularisation, said active fraction having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells.

84. Use of 7 S immunoglobulin or gamma globulin or a serum globulin fraction that is mainly composed of IgG molecules or intravenous immunoglobulin for the manufacture of a medicament for the treatment of choroidal neovascularisation in an amount effective to inhibit choroidal neovascularisation.

85. Use of an active fraction of 7 S immunoglobulin or gamma globulin or a serum globulin fraction that is mainly composed of IgG molecules or intravenous immunoglobulin for the manufacture of a medicament for the treatment of choroidal neovascularisation, wherein said active fraction is present in the medicament in an amount effective to inhibit choroidal neovascularisation.

86. Use of 7 S immunoglobulin or gamma globulin or a serum globulin fraction that is mainly composed of IgG molecules or intravenous immunoglobulin for the manufacture of a medicament for the treatment of choroidal neovascularisation, said 7 S immunoglobulin or gamma globulin or a serum globulin fraction that is mainly composed of IgG molecules or intravenous immunoglobulin having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said 7 S immunoglobulin or gamma globulin or a serum globulin fraction that is mainly composed of IgG molecules or intravenous immunoglobulin is present in the medicament in an amount effective to inhibit choroidal neovascularisation.

87. Use of an active fraction of 7 S immunoglobulin or gamma globulin or a serum globulin fraction that is mainly composed of IgG molecules or intravenous immunoglobulin for the manufacture of a medicament for the treatment of choroidal neovascularisation, said active fraction having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said active fraction is present in the medicament in an amount effective to inhibit choroidal neovascularisation.

88. Use of 7 S immunoglobulin or gamma globulin or a serum globulin fraction that is mainly composed of IgG molecules or intravenous immunoglobulin for the manufacture of a medicament for the treatment of choroidal neovascularisation, said 7 S immunoglobulin or gamma globulin or a serum globulin fraction that is mainly composed of IgG molecules or intravenous immunoglobulin having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said 7 S immunoglobulin or gamma globulin or a serum globulin fraction that is mainly composed of IgG molecules or intravenous immunoglobulin is present in the medicament in an amount effective to inhibit the proliferation of choroidal endothelial cells.

89. Use of an active fraction of 7 S immunoglobulin or gamma globulin or a serum globulin fraction that is mainly composed of IgG molecules or intravenous immunoglobulin for the manufacture of a medicament for the treatment of choroidal neovascularisation, said active fraction having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said active fraction is present in the medicament in an amount effective to inhibit the proliferation of choroidal endothelial cells.

90. Use of 7 S immunoglobulin or gamma globulin or a serum globulin fraction that is mainly composed of IgG molecules or intravenous immunoglobulin for the manufacture of a medicament for the treatment of choroidal neovascularisation, said 7 S immunoglobulin or gamma globulin or a serum globulin fraction that is mainly composed of IgG molecules or intravenous immunoglobulin having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said 7 S immunoglobulin or gamma globulin or a serum globulin fraction that is mainly composed of IgG molecules or intravenous immunoglobulin is present in the medicament in an amount effective to inhibit the migration of choroidal endothelial cells.

91. Use of an active fraction of 7 S immunoglobulin or gamma globulin or a serum globulin fraction that is mainly composed of IgG molecules or intravenous immunoglobulin for the manufacture of a medicament for the treatment of choroidal neovascularisation, said active fraction having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said active fraction is present in the medicament in an amount effective to inhibit the migration of choroidal endothelial cells.

92. Use of an active fraction of 7 S immunoglobulin or gamma globulin or a serum globulin fraction that is mainly composed of IgG molecules or intravenous immunoglobulin for the manufacture of a medicament for the treatment of choroidal neovascularisation, wherein said active fraction being selected from the group consisting of IgG₁, IgG₂, IgG₃, IgG₄ and mixtures thereof.

93. The use of claim 92, wherein said active fraction is composed of fragments of gamma immunoglobulin selected from the group consisting of F(ab′)₂, Fab′, Fab and Fc.

94. Use of an active fraction of 7 S immunoglobulin or gamma globulin or a serum globulin fraction that is mainly composed of IgG molecules or intravenous immunoglobulin for the manufacture of a medicament for the treatment of choroidal neovascularisation, said active fraction having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said active fraction is selected from the group consisting of IgG₁, IgG₂, IgG₃, IgG₄ and mixtures thereof.2

95. The use of claim 94, wherein said active fraction is composed of fragments of gamma immunoglobulin selected from the group consisting of F(ab′)₂, Fab′, Fab and Fc.

96. Use of an active fraction of 7 S immunoglobulin or gamma globulin or a serum globulin fraction that is mainly composed of IgG molecules or intravenous immunoglobulin for the manufacture of a medicament for the treatment of choroidal neovascularisation in an amount effective to inhibit choroidal neovascularisation and wherein said active fraction is selected from the group consisting of IgG₁, IgG₂, IgG₃, IgG₄ and mixtures thereof.

97. The use of claim 96, wherein said active fraction is composed of fragments of gamma immunoglobulin selected from the group consisting of F(ab′)₂, Fab′, Fab and Fc.

98. Use of an active fraction of 7 S immunoglobulin or gamma globulin or a serum globulin fraction that is mainly composed of IgG molecules or intravenous immunoglobulin for the manufacture of a medicament for the treatment of choroidal neovascularisation, said active fraction having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said active fraction is present in the medicament in an amount effective to inhibit choroidal neovascularisation and wherein said active fraction is selected from the group consisting of IgG₁, IgG₂, IgG₃, IgG₄ and mixtures thereof.

99. The use of claim 98, wherein said active fraction is composed of fragments of gamma immunoglobulin selected from the group consisting of F(ab′)₂, Fab′, Fab and Fc.

100. A method for the treatment of choroidal neovascularisation which comprises administering to the patient a preparation of 7 S immunoglobulin or gamma globulin or a serum globulin fraction that is mainly composed of IgG molecules or intravenous immunoglobulin.

101. A method for the treatment of choroidal neovascularisation which comprises administering to the patient a preparation of an active fraction of 7 S immunoglobulin or gamma globulin or a serum globulin fraction that is mainly composed of IgG molecules or intravenous immunoglobulin.

102. A method for the treatment of choroidal neovascularisation which comprises administering to the patient a preparation of 7 S immunoglobulin or gamma globulin or a serum globulin fraction that is mainly composed of IgG molecules or intravenous immunoglobulin in an amount effective to inhibit choroidal neovascularisation.

103. A method for the treatment of choroidal neovascularisation which comprises administering to the patient a preparation of an active fraction of 7 S immunoglobulin or gamma globulin or a serum globulin fraction that is mainly composed of IgG molecules or intravenous immunoglobulin in an amount effective to inhibit choroidal neovascularisation.

104. A method for the treatment of choroidal neovascularisation which comprises administering to the patient a preparation of 7 S immunoglobulin or gamma globulin or a serum globulin fraction that is mainly composed of IgG molecules or intravenous immunoglobulin, said 7 S immunoglobulin or gamma globulin or a serum globulin fraction that is mainly composed of IgG molecules or intravenous immunoglobulin having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells.

105. A method for the treatment of choroidal neovascularisation which comprises administering to the patient a preparation of an active fraction of 7 S immunoglobulin or gamma globulin or a serum globulin fraction that is mainly composed of IgG molecules or intravenous immunoglobulin, said active fraction having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells.

106. A method for the treatment of choroidal neovascularisation which comprises administering to the patient a preparation of 7 S immunoglobulin or gamma globulin or a serum globulin fraction that is mainly composed of IgG molecules or intravenous immunoglobulin, said 7 S immunoglobulin or gamma globulin or a serum globulin fraction that is mainly composed of IgG molecules or intravenous immunoglobulin having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said 7 S immunoglobulin or gamma globulin or a serum globulin fraction that is mainly composed of IgG molecules or intravenous immunoglobulin is present in the medicament in an amount effective to inhibit choroidal neovascularisation.

107. A method for the treatment of choroidal neovascularisation which comprises administering to the patient a preparation of an active fraction of 7 S immunoglobulin or gamma globulin or a serum globulin fraction that is mainly composed of IgG molecules or intravenous immunoglobulin, said active fraction having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said active fraction is present in the medicament in an amount effective to inhibit choroidal neovascularisation.

108. A method for the treatment of choroidal neovascularisation which comprises administering to the patient a preparation of 7 S immunoglobulin or gamma globulin or a serum globulin fraction that is mainly composed of IgG molecules or intravenous immunoglobulin, said 7 S immunoglobulin or gamma globulin or a serum globulin fraction that is mainly composed of IgG molecules or intravenous immunoglobulin having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said 7 S immunoglobulin or gamma globulin or a serum globulin fraction that is mainly composed of IgG molecules or intravenous immunoglobulin is present in the medicament in an amount effective to inhibit the proliferation of choroidal endothelial cells.

109. A method for the treatment of choroidal neovascularisation which comprises administering to the patient a preparation of an active fraction of 7 S immunoglobulin or gamma globulin or a serum globulin fraction that is mainly composed of IgG molecules or intravenous immunoglobulin, said active fraction having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said active fraction is present in the medicament in an amount effective to inhibit the proliferation of choroidal endothelial cells.

110. A method for the treatment of choroidal neovascularisation which comprises administering to the patient a preparation of 7 S immunoglobulin or gamma globulin or a serum globulin fraction that is mainly composed of IgG molecules or intravenous immunoglobulin, said 7 S immunoglobulin or gamma globulin or a serum globulin fraction that is mainly composed of IgG molecules or intravenous immunoglobulin having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said 7 S immunoglobulin or gamma globulin or a serum globulin fraction that is mainly composed of IgG molecules or intravenous immunoglobulin is present in the medicament in an amount effective to inhibit the migration of choroidal endothelial cells.

111. A method for the treatment of choroidal neovascularisation which comprises administering to the patient a preparation of an active fraction of 7 S immunoglobulin or gamma globulin or a serum globulin fraction that is mainly composed of IgG molecules or intravenous immunoglobulin, said active fraction having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said active fraction is present in the medicament in an amount effective to inhibit the migration of choroidal endothelial cells.

112. A method for the treatment of choroidal neovascularisation which comprises administering to the patient a preparation of an active fraction of 7 S immunoglobulin or gamma globulin or a serum globulin fraction that is mainly composed of IgG molecules or intravenous immunoglobulin, wherein said active fraction being selected from the group consisting of IgG₁, IgG₂, IgG₃, IgG₄ and mixtures thereof.

113. The method of claim 112, wherein said active fraction is composed of fragments of gamma immunoglobulin selected from the group consisting of F(ab′)₂, Fab′, Fab and Fc.

114. A method for the treatment of choroidal neovascularisation which comprises administering to the patient a preparation of an active fraction of 7 S immunoglobulin or gamma globulin or a serum globulin fraction that is mainly composed of IgG molecules or intravenous immunoglobulin, said active fraction having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said active fraction is selected from the group consisting of IgG₁, IgG₂, IgG₃, IgG₄ and mixtures thereof.

115. The method of claim 35, wherein said active fraction is composed of fragments of gamma immunoglobulin selected from the group consisting of F(ab′)₂, Fab′, Fab and Fc.

116. A method for the treatment of choroidal neovascularisation which comprises administering to the patient a preparation of an active fraction of 7 S immunoglobulin or gamma globulin or a serum globulin fraction that is mainly composed of IgG molecules or intravenous immunoglobulin in an amount effective to inhibit choroidal neovascularisation and wherein said active fraction is selected from the group consisting of IgG₁, IgG₂, IgG₃, IgG₄ and mixtures thereof.

117. The method of claim 116, wherein said active fraction is composed of fragments of gamma immunoglobulin selected from the group consisting of F(ab′)₂, Fab′, Fab and Fc.

118. A method for the treatment of choroidal neovascularisation which comprises administering to the patient a preparation of an active fraction of 7 S immunoglobulin or gamma globulin or a serum globulin fraction that is mainly composed of IgG molecules or intravenous immunoglobulin, said active fraction having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein active fraction is present in the medicament in an amount effective to inhibit choroidal neovascularisation and wherein said active fraction is selected from the group consisting of IgG₁, IgG₂, IgG₃, IgG₄ and mixtures thereof.

119. The method of claim 118, wherein said active fraction is composed of fragments of gamma immunoglobulin selected from the group consisting of F(ab′)₂, Fab′, Fab and Fc.

120. Pharmaceutical composition essentially consisting of an active fraction of 7 S immunoglobulin or gamma globulin or a serum globulin fraction that is mainly composed of IgG molecules or intravenous immunoglobulin as pharmaceutically active compound.

121. Pharmaceutical composition essentially consisting of an active fraction of 7 S immunoglobulin or gamma globulin or a serum globulin fraction that is mainly composed of IgG molecules or intravenous immunoglobulin as pharmaceutically active compound in an amount effective to inhibit choroidal neovascularisation.

122. Pharmaceutical composition essentially consisting of an active fraction of 7 S immunoglobulin or gamma globulin or a serum globulin fraction that is mainly composed of IgG molecules or intravenous immunoglobulin as pharmaceutically active compound, said active fraction having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells.

123. Pharmaceutical composition essentially consisting of an active fraction of 7 S immunoglobulin or gamma globulin or a serum globulin fraction that is mainly composed of IgG molecules or intravenous immunoglobulin as pharmaceutically active compound, said active fraction having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells.
wherein said active fraction is present in the medicament in an amount effective to inhibit choroidal neovascularisation.

124. Pharmaceutical composition essentially consisting of an active fraction of 7 S immunoglobulin or gamma globulin or a serum globulin fraction that is mainly composed of IgG molecules or intravenous immunoglobulin for the manufacture of a medicament for the treatment of choroidal neovascularisation, said active fraction having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said active fraction is present in the medicament in an amount effective to inhibit the proliferation of choroidal endothelial cells.

125. Pharmaceutical composition essentially consisting of an active fraction of 7 S immunoglobulin or gamma globulin or a serum globulin fraction that is mainly composed of IgG molecules or intravenous immunoglobulin as pharmaceutically active compound, said active fraction having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said active fraction is present in the medicament in an amount effective to inhibit the migration of choroidal endothelial cells.

126. Pharmaceutical composition essentially consisting of an active fraction of 7 S immunoglobulin or gamma globulin or a serum globulin fraction that is mainly composed of IgG molecules or intravenous immunoglobulin as pharmaceutically active compound, wherein said active fraction is one, two or three of the components selected from the group consisting of IgG₁, IgG₂, IgG₃, IgG₄.

127. The pharmaceutical composition of claim 126, wherein said active fraction is composed of fragments of gamma immunoglobulin selected from the group consisting of F(ab′)₂, Fab′, Fab and Fc.

128. Pharmaceutical composition essentially consisting of an active fraction of 7 S immunoglobulin or gamma globulin or a serum globulin fraction that is mainly composed of IgG molecules or intravenous immunoglobulin as pharmaceutically active compound in an amount effective to inhibit choroidal neovascularisation and wherein said active fraction is one, two or three of the components selected from the group consisting of IgG₁, IgG₂, IgG₃, IgG₄.

129. The pharmaceutical composition of claim 128, wherein said active fraction is composed of fragments of gamma immunoglobulin selected from the group consisting of F(ab′)₂, Fab′, Fab and Fc.

130. Pharmaceutical composition essentially consisting of an active fraction of 7 S immunoglobulin or gamma globulin or a serum globulin fraction that is mainly composed of IgG molecules or intravenous immunoglobulin as pharmaceutically active compound, said active fraction having the ability to inhibit one or more activities selected from the group consisting of:
a) choroidal neovascularisation,
b) proliferation of choroidal endothelial cells,
c) migration of choroidal endothelial cells,
d) tube formation of choroidal endothelial cells,
wherein said active fraction is one, two or three of the components selected from the group consisting of IgG₁, IgG₂, IgG₃, IgG₄.

131. The pharmaceutical composition of claim 130, wherein said active fraction is composed of fragments of gamma immunoglobulin selected from the group consisting of F(ab′)₂, Fab′, Fab and Fc.
